(19) United States
(12) Patent Application Publication
(10) Pub. No.: US 2021/0085945 A1
(43) Pub. Date: Mar. 25, 2021

(54) Non-invasive Transportation Method

(71) Applicant: Tze Guan ONG, Singapore (SG)

(72) Inventor: Tze Guan ONG, Singapore (SG)

(21) Appl. No.: 17/107,392

(22) Filed: Nov. 30, 2020

Related U.S. Application Data
(63) Continuation of application No. PCT/SG2019/050280, filed on May 30, 2019.

(30) Foreign Application Priority Data
May 30, 2018 (SG) 10201804566U

(51) Int. Cl. A61M 37/00

(52) U.S. Cl. CPC A61M 37/0015; A61M 37/0092; A61M 2037/0023; A61M 2037/0007; A61M 2202/06; A61M 2202/04; A61M 2037/0046

(57) ABSTRACT

Embodiments of the present invention provide a non-invasive transportation method which relates to the field of transdermal transportation and is aimed at improving absorption of active ingredients by skin. The non-invasive transportation method includes: applying needlelike crystals onto the skin, and forming a micro-channel array in epidermis by the needlelike crystals. The present invention is applicable to the transdermal transportation of active ingredients, and more commonly used in cosmetology processes.

### CROSS-REFERENCE TO EARLIER FILED APPLICATIONS

The present application claims the benefit of and is a continuation of application No. PCT/SG2019/050280 filed May 30, 2019, which claims the benefit of application No. SG 10201804566U filed May 30, 2018, the entire disclosures of which are hereby incorporated by reference.

### TECHNICAL FIELD OF THE INVENTION

The present invention relates to the field of transdermal transportation and in particular to a non-invasive transportation method. The method can be used in cosmetology and dermis regeneration.

### BACKGROUND OF THE INVENTION

As a person ages, functions of the dermis structure of his/her skin decline, since the skin loses collagen and elastin fibers due to slower production of crucial compounds which are required by the skin. To keep dermis regeneration at the best state, proper stimuluses and active substances are required.

Due to physical, environmental and physiological conditions, a person can also be inflicted with all kinds of disorders. It is highly possible that cures or treatments for such disorders may require the transdermal transport of compounds. As used herein, the term compound may refer to any substance that is to be applied onto, transported into, or transported through the skin. Such compounds may include, but are not limited to, active-ingredients, drugs, photosensitizers, viruses, etc.). Such compounds may be synthetic or naturally occurring, isolated or otherwise. It is also possible that these compounds are capable of entering the circulatory system and be transported throughout the body.

Due to presence of the stratum corneum of the epidermis, absorption of compounds by the skin is highly limited. The stratum corneum of the epidermis is a protective barrier formed by cells of superficial layers, and prevents macromolecular substances and organic substances from entering the skin and being absorbed by the body. Since many active substances cannot readily penetrate the dermis or deeper to reach blood vessels through the epidermis, the efficacy of dermis regeneration by topically applying an active substance is very poor.

To solve this problem, numerous invasive or non-invasive transdermal transportation techniques have been developed.

In the invasive transdermal transportation techniques, the epidermis is punctured by a device with small needles, and an active substance enters the skin through the formed puncture channels. In another form of the invasive transdermal transportation techniques, an active substance is directly injected into the dermis. These invasive transdermal transportation techniques may achieve good effects but could result in damage to the skin. If the damage is treated improperly, complications may result.

In the non-invasive transdermal transportation techniques, the penetration of active substances into the dermis through the stratum corneum may be via natural channels (e.g., those of sweat glands and hair follicles). Such penetration may be facilitated or enhanced by using various transportation techniques. Examples of the non-invasive transdermal transportation techniques include, but are not limited to, iontophoresis, electrophoresis, electroporation, water jetting, air jetting, micro-needle based device, ultrasound, laser dermabrasion and other dermabrasion treatments.

Although the non-invasive transdermal transportation techniques show deeper penetration of the active substances into the dermis, their effects are not as good as the invasive transdermal transportation techniques. This is because the active substances need to penetrate through the stratum corneum in the non-invasive transdermal transportation techniques, while the stratum corneum functions to prevent the penetration of the active substances.

### SUMMARY OF THE INVENTION

A main objective of one embodiment of the present invention is to provide a non-invasive transdermal transportation method which greatly improves efficacy of the transdermal transportation on the basis of the existing non-invasive transdermal transportation techniques.

To achieve this objective, embodiments of the present disclosure may employ the following technical solutions.

One embodiment of the disclosure relates to a non-invasive transportation method is provided, including:
applying penetrative means onto skin; and
forming a micro-channel array in the epidermis by the penetrative means, transporting a compound into the skin via the micro-channel array.

The “penetrative means” may comprise nano-sized or micron-sized particles, wherein each particle comprises at least one pointed end sufficient to cause abrasion, perturbation or penetration of a skin layer, e.g., the stratum corneum, epidermis or dermis layer. The pointed end may be an elongated protrusion, which may take on any shape suitable, e.g., round shaped, needle-shaped, tubular, columnar, conical shape, pyramidal, etc., provided that the protrusion is capable of causing abrasion, or penetration of a skin layer.

The particle may be elongated, e.g., having a needle-like shape, pin shape or tubular shape. The particle may be irregularly shaped and comprises a plurality of pointed edges or tips or protrusions. The penetrative means may be composed partially or fully of material which are biodegradable. For instance, the penetrative means may be selected to be degradable and absorbed by the human/animal skin/body. Where the penetrative means is not degradable or is only partially degradable, the remnant material after absorption by the skin may be removed via chemical means (e.g., solvent), or physical means (e.g., washing or physical extraction methods such as pulling out the embedded particles), or a combination thereof.

The size of the particle may not be particularly limited and may be selected based on the size of the compound to be administered to the skin. In general, the particle may have a width from about 0.3 μm to about 0.5 mm, whereas the length of the particle may be from about 20 μm to about 1 mm.

The particle may be a synthetic construct. The particle may also be naturally occurring or is isolated from a naturally occurring source.

When brought into contact with skin, the particle may increase the permeability of the stratum corneum by any one or more of the following means:
(1) creating breaks in the stratum corneum;
(2) embedding itself in and through the stratum corneum and deeper. In one embodiment, the penetrative means can be absorbed by the body over time. In other embodiments, the penetrative means may be removed physically or chemically or physio-chemically as they cannot be broken down and adsorbed by the body.

The penetrative means may be applied onto an area of the skin first prior to or after exposing the applied skin area to the compound intended for transdermal transportation.

Alternatively, the penetrative means may be mixed with the compound prior to application onto the skin surface.

The method may further include:
facilitating transportation of the compound into the skin in combination with one or more of other transdermal transportation methods,
wherein the other transdermal transportation methods include, but are not limited to, iontophoresis, electrophoresis, electroporation, water jetting, air jetting, micro-needle device, ultrasound, laser dermabrasion and other dermabrasion treatments, etc.

The method may include pressing or kneading the skin after application of the penetrative means and before applying the compound. Alternatively, the method may comprise pressing or kneading the skin after applying both the penetrative means and the compound. The disclosed method may be used in a cosmetology process or a regeneration technique.

In a non-invasive transportation method provided by one embodiment of the present invention, a micro-channel array is formed by applying crystals with a needle-like microstructure on a face of a subject, and transporting an active substance into or past the stratum corneum layer of said subject's skin through at least one of the channels of said micro-channel array. Advantageously, the penetration of an active substance through skin barrier is facilitated, and the skin is nourished deeply. Additionally, after penetrating into the skin, the micro-needle structure of the crystals can activate the vigor of the skin, enable the skin to generate collagen and other substances, and thus facilitate skin regeneration. Finally, by the naturopathy of the skin, the crystals in the skin are degraded and then absorbed by the skin.

### BRIEF DESCRIPTION OF THE DRAWINGS

To describe the technical solutions in the embodiments of the present invention or in the prior art more clearly, the accompanying drawings to be used in description of the embodiments or the prior art will be briefly described below. Apparently, the accompanying drawings described hereinafter are just for some of the embodiments of the present invention, and a person of ordinary skill in the art can obtain other drawings according to these drawings without any creative effort.

FIG. 1 is a sectional view of the skin structure;

Reference Numerals:
1—epidermis;
11—stratum corneum;
12—stratum lucidum;
13—stratum granulosum;
14—stratum spinosum;
15—stratum basale;
2—dermis;
21—Meissner's corpuscles;
22—capillary;
23—dermal papilla;
24—arrector pili muscle;
25—sebaceous gland;
3—subcutaneous tissue;
31—hair shaft;
32—sweat gland pore;
33—sweat gland duct;
34—sweat gland;
35—adipose tissue
36—arteriole;
37—venule;
38—hair papilla; and
39—lamellar corpuscle.

FIG. 2 is a picture of finished product of roller-type or a pressing-type micro-needle in the prior art;

FIG. 3 is a comparison diagram of the roller-type micro-needle, the pressing-type micro-needle, and Embodiment 1 of the present application, when applied to the skin;

FIG. 4 is a comparison diagram of the roller-type micro-needle, pressing-type micro-needle, and Embodiment 1 of the present application, after applied to the skin;

FIG. 5 shows needlelike crystals suspended in homogeneous liquid, observed with a microscope (100×);

FIG. 6 is a side view and a sectional view of the needlelike crystals; and

FIG. 7 shows the needlelike crystals observed with an optical microscope (10××40×).

FIG. 8 is a schematic drawing depicting one embodiment of the present invention, wherein a media comprising the penetrative means of the present invention is used in conjunction with a transdermal transportation technique.

FIGS. 9A and 9B are photographs showing the comparative depth of penetration of the active compounds on skin treated with the penetrative means (FIG. 9B) and on skin without such treatment (FIG. 9A).

FIG. 10 is a graph showing the effect on the penetration of the active compound with increasing duration of iontophoresis.

FIG. 11 is a graph showing the effect on the penetration of the active with increasing current applied during iontophoresis.

### DETAILED DESCRIPTION OF THE INVENTION

The technical solutions in the embodiments of the present invention will be clearly and completely described below with reference to the accompanying drawings in the embodiments of the present invention.

A non-invasive transportation method provided by one embodiment of the present invention will be described below with reference to the accompanying drawings.

In one non-limiting example of the present disclosure, crystals having a needlelike microstructure are used. After the crystals is applied on the skin, the needlelike crystals form a micro-channel array in epidermis, and an active ingredient is transported into the skin by virtue of the micro-channel array.

The microstructure of the crystals is needlelike and invisible to the naked eyes. The crystals are suspended in homogeneous liquid. FIG. 5 shows the microstructure of the crystals observed with a 100× microscope.

Combining with FIG. 1, it can be known that epidermis 1 of skin is composed of stratum corneum 11, stratum lucidum 12, stratum granulosum 13, stratum spinosum 14 and stratum basale 15. Dermis 2 of the skin includes Meissner's corpuscles 21, capillaries 22, dermal papillae 23, arrector pili muscles 24, sebaceous glands 25 and sweat gland ducts 33.

Depending on different ages, genders, photo-aging conditions, parts of body and the like of individuals, the thickness of the skin (the epidermis 1 and the dermis 2) varies widely. By taking facial skin as example, referring to Table 1, the depth of the facial skin (the epidermis 1 and the dermis 2) is between 0.5 mm to 1.5 mm. The thickness of corresponding facial epidermis 1 is between 0.05 mm to 0.2 mm, and the thickness of stratum corneum 11 is between 0.015 mm to 0.02 mm correspondingly. The stratum corneum 11 is the main skin barrier. For significant active substance penetration, certain means must be taken to allow the active substance to easily penetrate through the stratum corneum.

TABLE 1
Mean measured values of the thickness of skin

| | Subject A | Subject B | Subject C |
|---|---|---|---|
| Site | (mm) | (mm) | (mm) |
| Upper lip | 0.68 ± 0.09 | 1.01 ± 0.01 | 0.79 ± 0.16 |
| Lower lip | 0.78 ± 0.21 | 0.83 ± 0.07 | 0.85 ± 0.15 |
| Philtrum | 0.90 ± 0.08 | 0.83 ± 0.09 | 0.76 ± 0.09 |
| Jaw | 1.06 ± 0.10 | 1.24 ± 0.05 | 1.06 ± 0.11 |
| Upper eyelid | 0.41 ± 0.13 | 0.40 ± 0.06 | 0.32 ± 0.05 |
| Lower eyelid | 0.84 ± 0.06 | 1.04 ± 0.04 | 0.57 ± 0.05 |
| Forehead | 0.90 ± 0.13 | 1.16 ± 0.11 | 1.04 ± 0.04 |
| Right cheek | 1.04 ± 0.10 | 1.07 ± 0.06 | 1.11 ± 0.11 |
| Left cheek | 1.11 ± 0.09 | 1.20 ± 0.09 | 1.20 ± 0.04 |
| Malar eminence | 0.97 ± 0.07 | 1.62 ± 0.05 | 0.57 ± 0.04 |
| The submental | 1.06 ± 0.04 | 0.97 ± 0.05 | 0.65 ± 0.09 |
| Nasal cavity | 1.37 ± 0.14 | 1.17 ± 0.09 | 1.11 ± 0.06 |
| Nasal dorsum | 0.60 ± 0.06 | 0.79 ± 0.06 | 0.81 ± 0.09 |
| Right neck | 0.55 ± 0.09 | 0.25 ± 0.04 | 0.77 ± 0.07 |
| Left neck | 0.38 ± 0.04 | 0.43 ± 0.03 | 0.80 ± 0.05 |

Subject A was an 82-year-old female subject, subject B was a 51-year-old female subject, and subject C was a 78-year-old male subject.

After the crystals with the needlelike structure have been applied onto the skin, a dense and uniform micro-channel array having a depth of 0.02 mm to 0.5 mm is formed in the epidermis 1 of the skin. Preferably, the micro-channel array can be further deepened by other means, for example, up to 0.1 mm to 0.8 mm. The micro-channel array penetrates through the stratum corneum 11 which is the main part of the skin barrier, so that the further penetration of the active ingredient into the dermis 2 through the skin barrier is facilitated. It is to be noted that, if this transportation method is used together with massage or other means, the micros-channel array can be formed at a deeper level. That is, the array of the crystals can be deepened by massage.

After the crystals form the micro-channel array, the crystals can be completely absorbed by a natural defense mechanism of the body, generally within 12 hours to 48 hours. Preferably, the crystals can be completely absorbed by the skin within 6 hours to one day. There is not any damage to the skin.

Specific composition of the crystals will not be specifically limited in the present disclosure. Any crystals that can be absorbed by the skin within 2 hours to 14 days, and has a needlelike microstructure and no harm to the skin, may be used.

Such crystals can be extracted from the nature, for example, from plants and other organic sources (e.g., sea sponges). Or course, such crystals that have a needlelike structure and can be eventually degraded and absorbed by the skin can also be synthesized artificially. This will not be specifically limited herein.

The plant for extraction can be preferably plant species of Araceae. Needlelike crystals in such plant varieties (e.g., raphide crystals) mainly functions to give a certain painful sense from gnawing, to small insects or other creatures that eat roots, stems, leaves or the like of the plants, so as to protect the plants themselves. Such needlelike crystals have no toxic or side effect, and can be degraded and absorbed by organisms.

The size of the needlelike crystals is generally in the μm range and/or the nm range. For example, the width can be between 0.3 μm to 8 μm, and the length can be between 20 μm to 600 μm. Actual dimensions may depend on the sizes of the needlelike crystals obtained/isolated from the natural sources. Dimensions of such crystals may also be controlled where such crystals are to be synthesized artificially. The specific dimensions of such crystals are not particularly limited as long as the crystals can enter the skin without causing any invasive injuries.

To visually show the microstructure of the needlelike crystals, in addition to the needlelike crystals in the suspension observed with the 100× microscope shown in FIG. 5, also calcium oxalate raphides (needlelike crystals) extracted from roots of Lasia spinosa observed with an optical microscope (10××40×) are shown in FIG. 7.

Additionally, the so-called needlelike shape is merely an exemplary shape. Any elongated structure advantageous for penetration into the skin can be used. For example, in the lengthwise direction, it is preferably spindle-shaped or arrow-shaped. The cross-section can be hollow or solid, and in a variety of geometric shapes, for example, a shape having smooth curve periphery such as circle, ellipse or the like; or a shape having angled periphery such as square, polygon, triangle, elongated rectangle or the like. Due to the large length-to-width ratio of the needlelike crystals, the difference in cross-section shapes has little influence on ability of penetration into the skin, but the cross-section is still preferably circular.

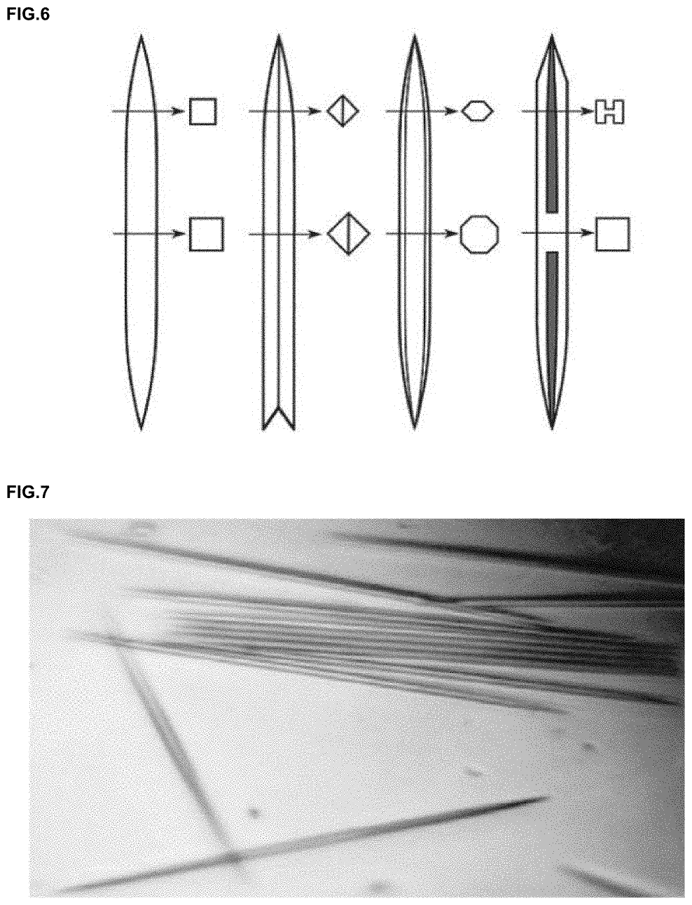

To describe the possible micro-shapes of the needlelike crystals better, some specific shapes of the needlelike crystals will be specifically exemplified below. As shown in FIG. 6, the structure of a needlelike crystal of type 1 looks like a spindle in the lengthwise direction, having two pointed ends and a thick middle portion, and its cross-section is a solid square. The structure of a needlelike crystal of type 2 looks like an arrow in the lengthwise direction, having a middle-arched top end and a cupped tail end, and its cross-section is a hollow rhombus having a middle interlayer. A needlelike crystal of type 3 looks like a spindle in the lengthwise direction, and its cross-section is a hollow polygon. The structure of a needlelike crystal of type 4 looks like a spindle in the lengthwise direction, its upper cross-section and lower cross-section are H-shaped, and its middle cross-section is a hollow square.

In addition to including single crystal, a needlelike crystal can be a needlelike crystal buddle formed by multiple single crystal clusters assembling, due to synthesis processes and natural extraction processes. When in use, such needlelike crystals enter the skin in unit of the needlelike crystal buddle.

In non-invasive transportation methods in the prior art, an active ingredient penetrates into the skin mainly by virtue of permeation. However, the permeation is weak, and most of the active ingredient is blocked outside the skin barrier, resulting in waste of the active ingredient. By this method, the needlelike crystals form a micro-channel array, and the active ingredient can penetrate through the skin barrier by the formed micro-channel array, so that the absorption of the active ingredient is facilitated.

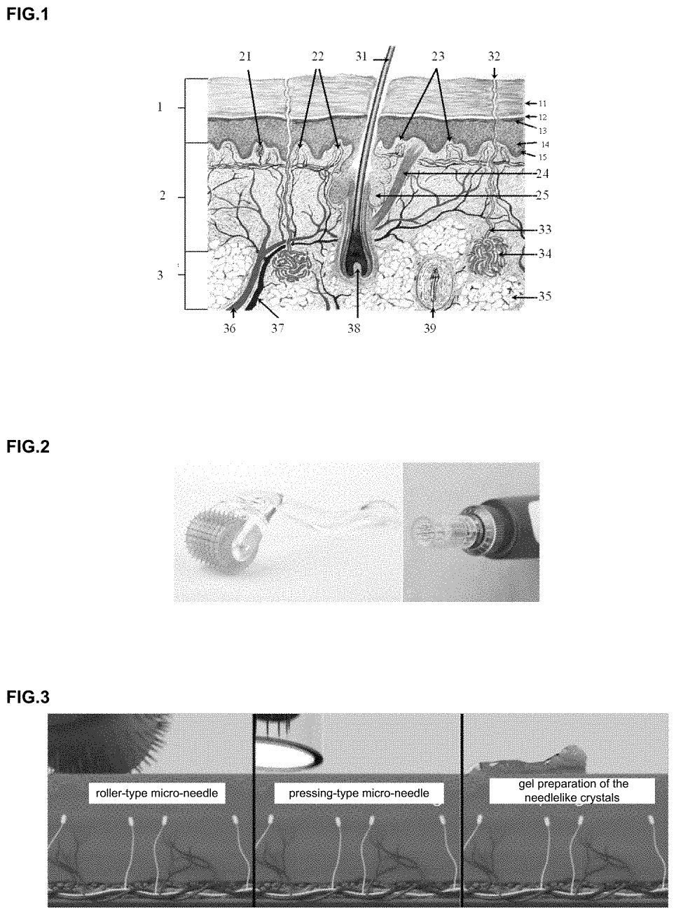

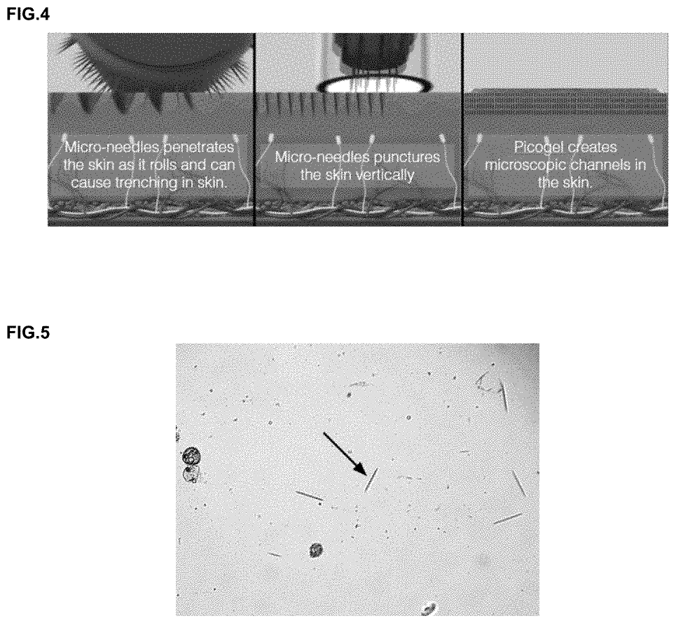

Compared with invasive transportation methods in the prior art, the crystals of the present application do not cause injuries to the skin, and the uniformity of the crystals distributing on the surface of the skin is much more improved in comparison with a micro-needle method in invasive cosmetology. FIG. 2 shows a picture of products of a roller-type micro-needle (left) and a pressing-type micro-needle (right) in the prior art. FIG. 3 shows a schematic diagram of the invasive roller-type micro-needle (e.g., Derma roller-type micro-needle) and pressing-type micro-needle in the prior art, and a non-invasive gel preparation of the needlelike crystals of the present application, before applied on the skin. FIG. 4 shows a schematic diagram of the three after applied on the skin. Both the roller-type micro-needle and the pressing-type micro-needle cause injuries to the skin. Moreover, since the diameter of needle tips in the micro-needle methods is approximately in millimeter scale and the distance between the needle tips is also in millimeter scale, the skin cannot be cared densely. For example, for the pressing-type micro-needle, the size of needle tips is large (0.5 mm to 1 mm) and the distance between needle tips is also large (e.g., 0.3 mm), leading to large areas of skin between the needle tips, and thus the skin cannot be completely and uniformly covered. However, the needlelike crystals of the present application do not cause injuries to the skin, and can form very dense micro-channels due to their microscopic size. It is to be noted that, to more visually show the arrangement of needlelike crystals, the needlelike crystals are drawn in tangible and dense arrangement. However, actually, the needlelike crystals need to be observed with a microscope and are not invisible to naked eyes.

In addition to facilitating the absorption of the active ingredient, during the penetration into the skin, the occurrence of the needlelike crystals also stimulate the immune system of the skin so that the skin generates new collagen and fibers to improve the dermis structure and increase toughness and elasticity of the skin.

The active ingredient can be mixed with the needlelike crystals and then applied on the skin; or, the needlelike crystals can be applied on the skin first, and then the active ingredient is applied thereafter. After the needlelike crystals are mixed with the active ingredient, homogeneous liquid may be obtained. The needlelike microstructure of the crystals is invisible to the naked eyes. The type of active ingredient will not be specifically limited in the present disclosure. For example, the active ingredient can be an active ingredient for cosmetology use or for medical use. The active ingredient for cosmetology use can have an effect of moisturizing, whitening, anti-aging or the like. Specifically, the active ingredient for cosmetology use can be hyaluronic acid, collagen, vitamin C, vitamin B3, aloe, arbutin, linolenic acid, botulinum toxin, hepatic cells, sheep placenta extract, human placenta extract, stem cell serum, various enzymes or the like. The active ingredient for medical use can be any medicines that can be transported through a transdermal transportation system in the prior art. The method of the present invention can be used alone or in combination with a transdermal transportation system in the prior art to realize better effects.

In addition, for dosage form of the product, the crystals can be combined with the active ingredient to form a liquid dosage form, a paste dosage form, a gel dosage form or the like by using processes in the prior art.

After the needlelike crystals are applied onto the skin, the absorption of the needlelike crystals can be facilitated by hand patting, massaging, kneading, pressing or other ways.

In addition, as a preferred embodiment of present invention, after the needlelike crystals and the active ingredient are applied, the absorption of the needlelike crystals can be further facilitated by other transdermal transportation techniques, for example, iontophoresis, electrophoresis, electroporation, water jetting, air jetting, micro-needle based device, ultrasound, laser dermabrasion and other dermabrasion treatments.

Both the iontophoresis and the electrophoresis are based on the action of electric field. The electric field acts on charged particles so as to non-invasively transport the active substance into the skin.

The electrophoresis may be performed for any duration necessary or required; subject to patient limits and desired absorption outcome. Optionally, the penetrating means may be replenished as frequently as needed during the course of electrophoresis. The electrophoresis can generally last for 5 minutes to 2 or 3 hours; preferably, 5 minutes to 60 minutes; further preferably, 10 minutes to 30 minutes; and, further preferably, 10 minutes to 20 minutes. In comparison with the prior art where only the electrophoresis is used, the electrophoresis in the present invention can be with the aid of the micro-channel array formed by the needlelike crystals, so that the absorption of the active ingredient is facilitated rapidly and greatly.

The electroporation is a cell transportation method. The cell membranes of cells are mainly composed of phospholipid molecules. The bilayer spatial structure of the phospholipid molecules can be instantaneously broken by proper electric current and then instantaneously recovers, so that the active substance easily enter the cell when the spatial structure is broken. This method maintains the integrated cell membrane and is a good non-invasive cell transportation method. The transportation method of the present invention can work together with the electroporation to facilitate the synergism of absorbing the active substance.

With regard to the water jetting, high-pressure water is used to impact the facial skin, so as to realize better penetration of the active substance into the skin through sweat glands; meanwhile, the thickness of the stratum corneum becomes smaller, so that it is more advantageous for the penetration of the active substance.

The principle of the air jetting is similar to that of the water jetting. High-pressure air acts on the surface of the skin so as to promote the penetration of the active substance.

In the ultrasound method, liquid is provided on the face and then ultrasonically treated to allow tiny bubbles to generate in the liquid. At the moment of the tiny bubbles bursting, the penetration of the active ingredient into the skin can be promoted.

The laser dermabrasion and other dermabrasion treatments are mainly to thin the stratum corneum so as to further facilitate the penetration of the needlelike crystals and the active substance.

All these methods can be used when applying the needlelike crystals and/or the active ingredient, to lead to cooperative synergism.

One embodiment of the present disclosure relates to a method for transdermal delivery of compounds, the method comprising the use of penetrative means with at least one transdermal delivery technique; and adjusting the strength of said transdermal delivery technique to control the delivery of said compounds.

In one embodiment, there is provided a method of transporting a compound across a skin, said method comprising the steps of: (a) puncturing at least an area of the skin with penetrative means to provide an array of microchannels extending partially or completely through an epidermis layer of the skin; and (b) administering, to said skin, a transdermal delivery technique comprising heating, ultra-sound, laser dermabrasion, air jetting, water jetting, electrophoresis, electro-osmosis, iontophoresis, or electroporation, wherein steps (a) and (b) are performed sequentially, or concurrently.

The disclosed method may be used solely for cosmetic purposes.

The method may comprise sequentially applying a composition comprising said compound in admixture with said penetrative means topically on a skin surface, followed by performing the transdermal delivery technique on the skin surface. The sequence may be reversed or repeated as needed.

Alternatively, the method may comprise applying the composition comprising the compound and the penetrative means on a skin surface, with concurrent application of the transdermal delivery technique on the skin surface.

The transdermal delivery technique may include but are not limited to one or more techniques as disclosed herein, e.g., heating, ultra-sound, laser dermabrasion, air jetting, water jetting, electrophoresis, electro-osmosis, iontophoresis, and electroporation. In one embodiment, the transdermal delivery technique is selected to be iontophoresis.

The strength, intensity and/or duration of the iontophoresis treatment may be adjusted to control the delivery of said active ingredient through the stratum corneum layer. For instance, during iontophoresis, the strength of the electric current being applied may be adjusted from about 1 mA to about 10 mA. It has been found that increasing the strength of the current may allow a larger concentration of the compound to penetrate the skin surface. In one embodiment, increasing the current from 2.5 mA to 5 mA and to 7.5 mA has been found to improve penetration of the compound by about 5% to about 35%. The improvement in penetration may also depend on the chemical composition and size of the compound being transported. Increasing the current strength may also affect 1) the time taken to complete transdermal transfer; (2) the proportion/fraction or quantity of the compound that may be transported transdermally; (3) the depth of the skin layer to which the compound may reach; and (4) the amount penetration through skin to subcutaneous level. The combination of iontophoresis with the use of penetrative means disclosed herein is particularly advantageous because precise control of the compound delivery can be achieved whereas such control was hitherto not contemplated or considered possible with existing use of iontophoresis. Advantageously, the disclosed combination may now provide means to deliver a precise dosage of the compound, to one or more specific target areas, at precise depths, to achieve the desired therapeutic or cosmetic outcome.

The penetrative means may comprise needle-like structures such as those described herein. The needle-like structures may also comprise naturally occurring (and optionally provided in isolated form) and/or synthetic needle-like structures. The needle-like structures may comprise both organic and inorganic compounds. Non-limiting examples of such needle-like structures may include, but are not limited to, raphides, sponge spicules, hyaluronic acid, etc.

The size of the penetrative means may be selected in order to cause micro-punctures in the skin layer to provide an array of micro-channels as described herein. The particles of the penetrative means may be selected in order to puncture the skin to cause the formation of micro-channels having a width of less than 0.1 to 100 microns and a depth of around 0.1 to 1 mm.

Advantageously, the intensity of the electric field, current or voltage applied, and/or the duration of the iontophoresis treatment, may be varied in order to achieve a desired rate of penetration of the active ingredient. The intensity of the transdermal delivery technique may also be adjusted in order to control the depth of penetration, and/or the amount of active ingredient that is being transported across the stratum corneum layer. The duration of the treatment may be from about 0.5 minutes to 10 minutes or from about 1 to 5 minutes. Increasing the duration of treatment may result in improved penetration of the compound. The duration may not be particularly limited and may depend on the skin's receptivity to the compound and/or the tolerance of the subject being treated and/or to achieve a desired therapeutic outcome.

In another embodiment, the present disclosure relates to a method for transdermal delivery of compounds, the method comprising the combinatory use of penetrative means as disclosed herein with at least two transdermal delivery techniques. In particular, the method may comprise applying a composition comprising said compound in admixture with penetrative means topically on a skin surface, and performing iontophoresis and electroporation, whether concurrently or sequentially, on the skin surface. Advantageously, the combination of these two techniques was found to synergistically improve the uptake of the compounds by the human/animal body. It is postulated that iontophoresis aids to improve the penetration of the compound into the skin, whereas electroporation aids to improve the penetration of the compound into the cells.

In another embodiment, the present disclosure relates to a media comprising the penetrative means of the present disclosure. The penetrative means may comprises means capable of puncturing the skin surface of a subject to thereby provide an array of microchannels extending partially and/or completely through the epidermis layer of the skin. The media may be a solid, liquid or a mixture (suspension). When provided as a solid media, the media may be flexible or non-flexible (rigid) and may comprise synthetic materials, organic materials or a combination of both. The media may be a gel, paste or an emulsion. The media may be fiber-woven, polymer-based, or gel-based. When provided as a cream or a gel, the media may cure or solidify under ambient conditions without further stimuli. The media may also be photo-curable or heat-curable or both. The media may be composed of a material which hardens/solidifies in the presence of other chemicals or water. In embodiments, the media may be in powder form, gel form or liquid form, which can “harden” into like a solid or soft plastic like material when combined with other chemicals, or when mixed with water. In one embodiment, the penetrative means may be admixed with the media (in powder form). Water may be added into the mixture to form a paste prior to application on the skin. The paste may harden into a rubber-like, gel-like or plastic-like layer, which can be removed by peeling it off the skin. While the mixture or paste is applied onto the skin layer, it may be physically agitated, e.g., rubbing or kneading, to cause abrasion/puncture of the skin. The mixture or paste may thereafter harden or solidify into a mask-like structure, which can be physically removed e.g., by peeling. In one embodiment, such a media may be suitable for use with penetrative means which may not be absorbed into skin layer. Advantageously, this allows the penetrative means to be removed from the skin layer at the time of removing the mask-like structure.

The media may optionally include one or more compounds mixed, dissolved or suspended therein. Where the media is a powder composition, the compounds may be form an admixture with the media prior to forming a paste. Alternatively, the compounds may be applied separately on the skin prior to the application of the paste.

The media may comprise a flexible mask capable of adopting the contours of a curved surface (e.g., skin on a face) upon contact. The penetrative means may be adhered to, attached to, embedded in, or impregnated on the media. The media may also be rolled, formed, molded, 3-D printed or casted from a mixture comprising the penetrative means, e.g., needle-like structures. The media may comprise organic fibers having the compound and/or the penetrative means coupled thereon. In one embodiment, the media may comprise one or more such sheets of organic fibers. In one embodiment, the media comprises a fiber mesh or woven fiber, e.g., a facial mask or skin pads. The media may comprise an adhesive layer that is capable of immobilizing the media on the skin surface upon contact.

When in use, the media may be applied onto a skin surface to bring the penetrative means in contact with the skin surface. The contact may be sufficiently close to allow the penetrative means to puncture the skin surface. Optionally, the media may be physically agitated when on the skin surface to generate abrasion between the media and the skin surface, e.g., including but not limited to, kneading, rubbing, pressing, or sonication. The physical agitation may be useful to cause abrasion and/or to generate an array of micro-channels as disclosed herein on the skin surface.

The media may be used on the skin surface prior to the application of a compound thereon. The media may be optionally retained on the skin surface during the application of the compound. Alternatively, the media may already comprise the compound intended for transdermal delivery. Further alternatively, the compound may be first applied to the skin, prior to contacting the skin with the media.

Advantageously, the media comprising the penetrative means may be useful to provide enhanced transdermal penetration of compounds. The provision of the penetrative means on a media may advantageously result in a more uniform array of micro-channels being formed on the skin surface due to the homogenous dispersion/distribution of the penetrative means within the media.

The media may also be used in combination with the methods disclosed in alternative embodiments 1 and/or 2.

In another embodiment, the present disclosure relates to a method for the transdermal delivery of a compound, the method comprising contacting a media as described herein with a skin surface; and applying at least one transdermal delivery technique disclosed herein to the skin. The transdermal delivery technique may include but is not limited to ionotophoresis, electrophoresis, electroosmosis, heating, ultrasonic treatment, radio frequency treatment, photo therapy, or electroporation. In one embodiment, the transdermal delivery technique is ionotophoresis.

The present disclosure further relates to a method of transporting a compound across a skin surface applying to a section of said skin a media as described above, to thereby puncture said skin with the penetrative means to provide an array of microchannels extending partially or completely through the epidermis layer of the skin; and administering, to said skin, at least one transdermal delivery technique selected from the group comprising heating, ultra-sound, laser dermabrasion, air jetting, water jetting, electrophoresis, electro-osmosis, iontophoresis, or electroporation, wherein steps (a) and (b) are performed sequentially, or concurrently. The method may be used solely for cosmetic purposes.

The transdermal delivery technique may be applied sequentially to the step of contacting the skin with said media. In particular, the media may be first contacted with a surface of the skin, and wherein the media may be optionally agitated; the method may be followed by the application of the transdermal delivery technique. The transdermal delivery technique may also be applied concurrently when contacting the skin with said media. Optionally, an additional step of applying the compound to the skin surface may be provided, depending on whether the media already comprises the compound.

The transdermal delivery technique may be applied on the skin surface, while the media remains in contact with the skin. Alternatively, the transdermal delivery technique may be applied after the media has been removed from the skin surface, exposing an area of abraded and punctured skin.

In another embodiment, the present disclosure relates to a method for the transdermal delivery of a compound, the method comprising contacting a media as described in Embodiment 3 with a skin surface; and applying at least one transdermal delivery technique disclosed herein to the skin; wherein the strength, intensity and/or duration of the transdermal delivery technique is adjusted to control the delivery of the active ingredient.

The transdermal delivery technique may be selected from ionotophoresis, electrophoresis, electroosmosis, or electroporation. In one embodiment, the transdermal delivery technique is ionotophoresis. Advantageously, the intensity of the electric field, current or voltage applied may be varied in order to achieve a desired rate of penetration of the active ingredient. In one embodiment, the strength of the electric current and the duration of treatment may be independently and/or concurrently adjusted to obtain a desired penetration profile. The intensity of the transdermal delivery technique may also be adjusted in order to control the depth of penetration, and/or the amount of active ingredient that is being transported through the stratum corneum layer.

The transdermal delivery technique may be applied sequentially to the step of contacting the skin with said media. In particular, the media may be first contacted with a surface of the skin, and optionally agitated; followed by the application of the transdermal delivery technique. The transdermal delivery technique may also be applied concurrently when contacting the skin with said media. Optionally, an additional step of applying the compound to the skin surface may be provided, depending on whether the media already comprises the compound.

The transdermal delivery technique may be applied on the skin surface, while the media remains in contact with the skin. Alternatively, the transdermal delivery technique may be applied after the media has been removed from the skin surface, exposing an area of abraded and punctured skin.

In another embodiment, the present disclosure relates to a method for the transdermal delivery of a compound, the method comprising contacting a media as described above with a skin surface; and applying at least two transdermal delivery techniques as disclosed herein to the skin; wherein the at least two transdermal delivery techniques are selected from ionotophoresis and electroporation.

The iontophoresis and electroporation may be performed concurrently or sequentially on the skin surface. Advantageously, the combination of these two techniques was found to synergistically improve the uptake of the compounds by the human/animal body. It is postulated that iontophoresis aids to improve the penetration of the compound into the skin, whereas electroporation aids to improve the penetration of the compound into the cells.

The transdermal delivery techniques may be applied after the step of contacting the skin with said media. In particular, the media may be first contacted with a surface of the skin, and wherein the media is optionally agitated; the method is thereafter followed by the application of the transdermal delivery techniques, whether applied in combination or sequentially.

The transdermal delivery techniques may be applied when contacting the skin with said media or when agitating the media, or both. Optionally, an additional step of applying the active ingredient to the skin surface may be provided, depending on whether the media already comprises the active ingredient.

The transdermal delivery techniques may be applied on the skin surface, while the media remains in contact with the skin. Alternatively, the transdermal delivery techniques may be applied after the media has been removed from the skin surface, exposing an area of abraded and punctured skin.

The present disclosure may further relate to a device for transporting a compound across a skin layer, said device comprising: (i) a media for application on the surface of said skin layer, said media comprising the compound to be transported, and a plurality of penetrative means configured to puncture said skin layer to thereby provide an array of microchannels extending partially or completely through an epidermis layer of the skin; and (ii) a stimulation means coupled to said skin or said media, said stimulation means configured to apply to said skin or media at least one transdermal delivery technique selected from the group consisting of heating, ultra-sound, laser dermabrasion, air jetting, water jetting, electrophoresis, electro-osmosis, iontophoresis, and electroporation.

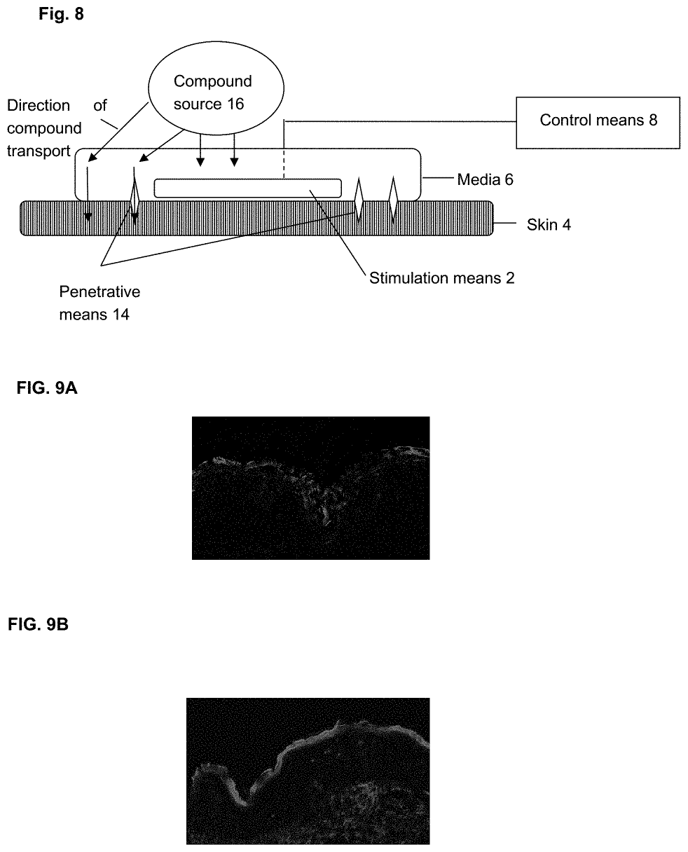

The present invention further contemplates the provision of a media 6 as described in the previous embodiments, wherein the media further comprises at least one stimulation means 2 incorporated therein as shown in FIG. 8. The stimulation means 2 may be configured to perform an enhancement technique as described herein, including but not limited to, heating, application of electric field, physical agitation, electroporation, and electrophoresis. The media 6 having a stimulation means 2 may be provided as a portable or wearable device. In one embodiment, the device may be a patch designed to adhere to the skin 4 surface.

The area of application of the device/patch is not particularly limited and depends on the desired location for topical delivery of drugs/active ingredients. For instance, the area of application may be face, eyes bags, eye lids, nose, arms, legs, back, torso, scalp, etc.

The media may optionally comprise at least one adhesive layer that is contacted with the skin 4 surface to thereby secure the media 6 to the skin surface. The adhesive layer may form a complete intermediate layer between the media and skin. Alternatively, the adhesive layer may be disposed on a partial surface of the media.

The device may be optionally coupled to a control means 8 configured to adjust and operate the stimulation means 2 (e.g., adjusting current strength, intensity and/or duration of electrophoresis). The media 6 may be optionally provided with the penetrative means 14 embedded within the media. Alternatively, the media 6 may be applied to an area of the skin where the penetrative means 14 has already been applied. Optionally, the media 6 may be coupled or communicated with a compound source 16 configured to supply the compound intended for transdermal delivery to the media 6. The compound source 16 may comprise means for controlling or regulating the compound flow rate, quantity of compounds supplied, and/or the duration of the supply. The compound source 16 may be configured to convey the compounds to the media for permeation onto the skin 4 or the compound source 16 may be configured to convey the compounds directly onto the skin layer. The compound may permeate the media 6 structure and get transported into the skin layer via simple diffusion or transport via the micro-channels created by the penetrative means 14. The presence of the stimulation means 2 may further enhance said transport. The supply of the compound may be a continuous supply or the compound may be supplied batchwise from the compound source 16.

Based on the foregoing principles of the present application, the non-invasive transportation method provided by the present application can be used in various cosmetology processes. Optionally, if the active ingredient is a medical ingredient, the non-invasive transportation method can also be used in therapeutic process. In conclusion, the present application is devoted to provide a non-invasive transportation method which can be used in various fields where it is necessary to facilitate the absorption of a substance through the skin.

To describe the non-invasive transportation method provided by the present application better, description will be given below by specific examples.

### Example 1

Cosmetics, oil and dust on subjects' faces were cleaned with cleansing emulsion and cleaning gel. An active ingredient was applied onto each subject's face for multiple times, 1 mL every time, specifically as follows:
a. 1 mL of the active ingredient was applied onto facial skin to uniformly and completely cover the facial skin;
b. when the facial skin became dry as the active ingredient thereon was absorbed and evaporated, another 1 mL of the active ingredient was applied onto the facial skin to uniformly and completely cover the facial skin;
c. steps a and b were repeated until total 5 mL of the active ingredient was used up; and
d. total time from the beginning to the end of applying the active ingredient was recorded.

### Comparative Example 1

The subjects were treated according to steps described in the following comparative example 1 five days later after the treatment described in Example 1, to avoid the interference of the previous test to the next test and ensure skin condition in Example 1 is most approximate to that in comparative example 1.
1. Cosmetics, oil and dust on the subjects's faces were cleaned by cleansing emulsion and cleaning gel.
2. 1 mL of needlelike crystals was applied onto each face, and then the face was strongly massaged for 1 minute.
3. The active ingredient was applied onto each subject's face for multiple times, 1 mL every time, specifically as follows:
a. 1 mL of the active ingredient was applied onto facial skin to uniformly and completely cover the facial skin;
b. when the facial skin became dry as the active ingredient thereon was absorbed and evaporated, another 1 mL of active ingredient was applied onto the facial skin to uniformly and completely cover the facial skin;
c. steps a and b were repeated until total 5 mL of the active ingredient was used up; and
d. total time from the beginning to the end of applying the active ingredient was recorded.

Note: the active ingredient in this test was stem cell extract.

The total time required to use up all the 5 mL of the active ingredient on each of the five subjects is listed in Table 2.

TABLE 2
Time for absorption of the active ingredient

| | |
|---|---|
| Example 1 | Comparative example 1 |

| | Absorption | | Absorption |
|---|---|---|---|
| Subject No. | time (min) | Subject No. | time (min) |
| Subject 1 | 18 | Subject 1 | 12 |
| Subject 2 | 16 | Subject 2 | 12 |
| Subject 3 | 16 | Subject 3 | 10 |
| Subject 4 | 20 | Subject 4 | 13 |
| Subject 5 | 18 | Subject 5 | 10 |
| Total | 88 | Total | 57 |
| absorption | | absorption | |
| time | | time | |
| Average | 17.6 | Average | 11.4 |
| absorption | | absorption | |
| time | | time | |

It can be observed from Table 2 that, for the subjects treated with the needlelike crystals, the absorption time of the active ingredient was obviously shortened by about 35.2%. It was demonstrate that the needlelike crystals improved the ability of the skin in the absorption of the active ingredient.

### Example 2

Cosmetics, oil and dust on subjects' faces were cleaned by cleansing emulsion and cleaning gel. A reagent bottle containing 20 mL of the active ingredient was connected to a roller of a TMT device. The TMT device was turned on and set at 80% power. A rolling actuator annularly acted on the whole facial skin of each subject. The time from the beginning of applying the active ingredient to the moment when the active ingredient was completely used up was recorded.

### Comparative Example 2

The subjects were treated by steps described in the following comparative example 2 five days later after the treatment described in Example 2, to avoid the interference of the previous test to the next test and ensure the skin condition in Example 2 is most approximate to that in comparison example 2.
1. Cosmetics, oil and dust on the subjects' faces were cleaned by cleansing emulsion and cleaning gel.
2. 2 mL of the needlelike crystal was applied onto each face, and then the face was strongly massaged for 1 minute.
3. A reagent bottle containing 20 mL of the active ingredient was connected to a rolling actuator of a TMT device.
4. The TMT device was turned on and set at 80% power.
5. The rolling actuator annularly acted on the whole facial skin of each subject.
6. The time from the beginning of applying the active ingredient to the moment when the active ingredient was completely used up was recorded.

Note: the active ingredient in this test was stem cell extract. The TMT device, from Mesoestetic, is a device with multiple functions of electrophoresis and electroporation.

The total time required to use up all the 20 mL of the active ingredient on the facial skin of each of the five subjects is listed in Table 3.

TABLE 3
Time for absorption of the active ingredient

| | |
|---|---|
| Embodiment 2 | Comparison example 2 |

| | Absorption | | Absorption |
|---|---|---|---|
| Subject No. | time (min) | Subject No. | time (min) |
| Subject 1 | 42 | Subject 1 | 18 |
| Subject 2 | 46 | Subject 2 | 23 |
| Subject 3 | 38 | Subject 3 | 19 |
| Subject 4 | 34 | Subject 4 | 14 |
| Subject 5 | 45 | Subject 5 | 21 |
| Total | 205 | Total | 95 |
| absorption | | absorption | |
| time | | time | |
| Average | 41 | Average | 19 |
| absorption | | absorption | |
| time | | time | |

It can be known from Table 3 that, for the subjects treated with the needlelike crystals, the absorption time of the active ingredient was obviously shortened by about 53.7%. It was proved that the needlelike crystals improved the ability of the skin in the absorption of the active ingredient.

Moreover, it is found from the comparison between Example 1 and Example 2 that, using the conductance technique to cooperate with the needlelike crystals, more active ingredient was absorbed within a nearly identical period of time. Thus, by the cooperative use of the needlelike crystals and the common transportation methods in the prior art, the synergistic effect can be realized.

### Example 3

In this example, 100 μL of hyaluronic acid (HA) conjugated with a dye compound (Cy5) is applied onto a skin surface alone (“comparative embodiment) and with the penetrative means of the present disclosure provided in gel form (“inventive embodiment”). In both embodiments, after application of the HA-Cy5, ionotophoresis was performed at 5 mA for 1 minute. The depth of penetration is then investigated via dye detection. It is observed that when the penetrative means of the present disclosure is applied, HA was detectable at the dermis region whereas in the comparative embodiment, HA was only detectable at the epidermis region.

| | Comparative | Inventive |
|---|---|---|
| | embodiment | Embodiment |
| Compound | Hyaluronic acid | Hyaluronic acid |
| | (2.2 MDa) with | (2.2 MDa) with |
| | dye (Cy5) | dye (Cy5) |
| Penetrative | Absent | Raphide crystals |
| means | | |
| Enhancement | Iontophoresis | Iontophoresis |
| technique | at 5 mA for 1 | at 5 mA for 1 |
| | minute | minute |
| Result | See FIG. 9A | See FIG. 9B |
| Depth of | Epidermis region | Dermis region |
| penetration | | |

As can be observed from the phosphorescent photo result in example 3, when the inventive method was applied, there is a significant “dark zone” occurring between the two glowing areas (HA-dyed). It may be surmised that that the dye-conjugated HA was “pushed” far beyond the dermis (not observed when the penetrative means was absent). It is postulated that the depth and amount of compound being delivered can be further controlled by appropriate adjustment of the current strength or modifying the duration of applying the iontophoresis technique.

### Example 4

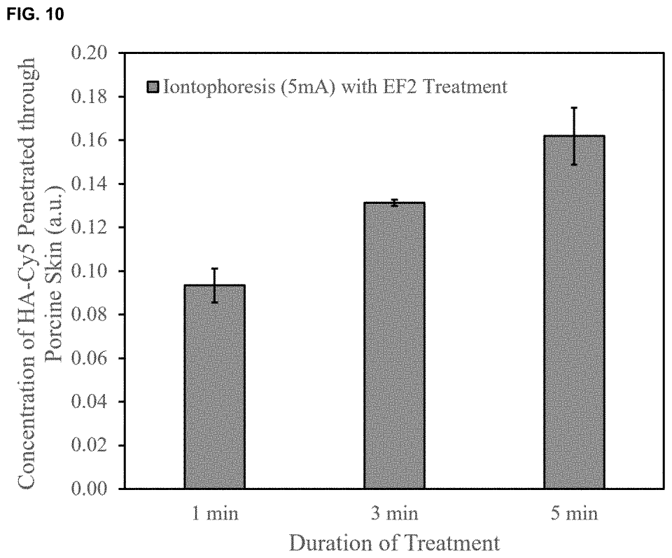

This example shows the effect of varying the duration of treatment on the penetration of the compound. In particular, this example compares the concentration of HA conjugated with Cy5 that has penetrated through the skin that has been treated with the penetrative means of the present invention and combined with iontophoresis under application durations of 1 minute, 3 minutes and 5 minutes respectively. FIG. 10 shows that the result of increasing the treatment duration from 1 to 5 minutes has the effect of increasing the amount of hyaluronic acid penetrating past the skin layer.

### Example 5

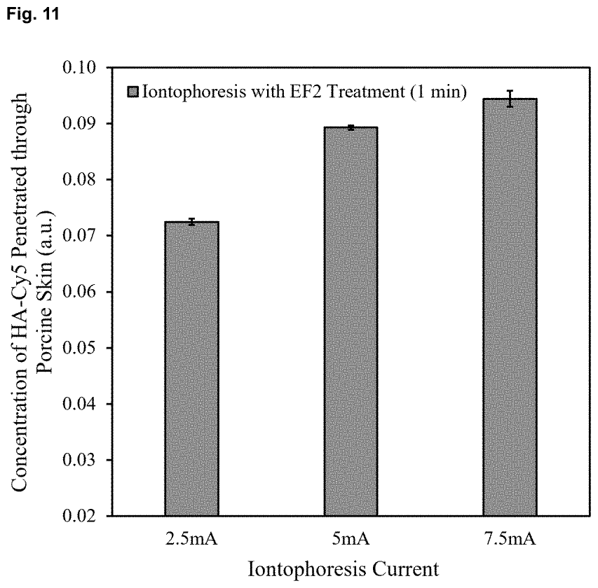

This example shows the effect of increasing the current intensity of the enhancement technique (ionotophoresis) on the penetration of the compound (HA-Cy5). It can be observed from FIG. 11 that increasing current strength has the effect of increasing the total quantity of compounds penetrating the skin.

The foregoing descriptions merely show specific implementations of the present invention, and the protection scope of the present invention is not limited thereto. A person of skill in the art can readily conceive of variations or replacements within the technical scope disclosed by the present invention, and these variations or replacements shall fall into the protection scope of the present invention. Accordingly, the protection scope of the present invention shall be subject to the protection scope of the claims.

### INDUSTRIAL APPLICATION

The above disclosed methods for transdermal delivery of compounds into and across the skin layer has practical industrial applications. These applications may include purely cosmetic uses which are non-therapeutic in nature, e.g., for aesthetic treatments, delivery of botulinum toxin, etc. Alternatively, it is also envisioned that the disclosed methods may be employed in therapeutic applications. These applications may include the treatment of dermatological disorders, e.g., eczema, psoriasis, skin cancer, etc. The disclosed methods may also be applied for the treatment of diseases in general, wherein the topical delivery of medicaments and drugs is required or preferred. In particular, the disclosed methods are expected to be useful for any therapeutic or cosmetic application which involves the subcutaneous administration/delivery of compounds into the epidermis or dermis layer. The disclosed methods may also be useful for the delivery of compounds (e.g., drugs) to the micro-circulatory system through the skin, wherein these compounds may eventually be transported into the circulatory system of the body. Accordingly, the methods disclosed herein are not limited to use in cosmetic procedures but rather, are expected to be useful for any type of therapeutic application which requires the transdermal/topical delivery of pharmaceuticals, or subcutaneous administration. The disclosed methods may also substitute conventional intravenous drug delivery by transporting the drugs into the blood circulation via transdermal delivery. Even further, the disclosed methods and penetrative means may be potentially used in gene therapy, e.g., for improving the transportation of the genes to the required sites for transfecting genetic material into cells. For instance, the disclosed methods may be used for transdermal delivery of RNA to cells in the skin. Such applications may see utility in the treatment of skin disorders, e.g., skin cancer. In exemplary embodiments, the genetic material may be delivered (e.g., by injection or topical application) at a site close to the target cells. The delivery of the genetic material to the target cells may be aided by the penetrative means of the present disclosure. Concurrently, the uptake of the genetic material by the target cells may be synergistically enhanced by the use of existing gene transfection techniques, e.g., electroporation.

1. A method of transporting a compound across a skin, said method comprising the steps of:
(a) puncturing at least an area of the skin with penetrative means to provide an array of microchannels extending partially or completely through an epidermis layer of the skin; and
(b) administering, to said skin, transdermal delivery techniques comprising a combination of iontophoresis and electroporation,
wherein steps (a) and (b) are performed sequentially, or concurrently.

2. The method of claim 1, wherein said penetrative means comprises needle-like structures.

3. The method of claim 1, wherein said penetrative means is isolated from a natural source.

4. The method of claim 1, wherein said array of microchannels comprises a plurality of channels extending completely or partially through the epidermis layer, each channel having, independently, a width of 0.1 to 100 microns and a depth of about 0.1 to 1 mm.

5. The method of claim 1, further comprising a step of adjusting the strength, intensity, and/or duration of the transdermal delivery techniques.

6. The method of claim 1, wherein said step (b) comprises administering to the skin at least one further transdermal delivery technique selected from the group consisting of heating, ultra-sound, laser dermabrasion, air jetting, water jetting, electrophoresis, and electro-osmosis.

7. A media comprising,
penetrative means capable of providing an array of microchannels extending through partially or completely an epidermis layer of the skin; and
a photocurable or a heat-curable polymer in admixture with said penetrative means.

8. The media of claim 7, being formulated as a solid, liquid, or a mixture, preferably selected from a gel, paste, or an emulsion.

9. The media of claim 7, further comprising at least one compound intended to be transported across the epidermis layer of said skin.

10. The media of claim 7, wherein the media is coupled to a stimulation means, said stimulation means being configured to perform at least one transdermal delivery technique selected the group consisting of heating, application of electric field, physical agitation, iontophoresis, electroporation, and electrophoresis.

11. The media of claim 10, wherein the stimulation means is configured to perform a combination of iontophoresis and electroporation.

12. The media of claim 7, wherein said penetrative means comprises needle-like structures.

13. The media of claim 7, wherein said penetrative means is isolated from a natural source.

14. A method of transporting a compound across a skin, said method comprising the steps of:
(a) applying to a section of said skin a media, to thereby puncture said skin with the penetrative means to provide an array of microchannels extending partially or completely through an epidermis layer of the skin; wherein i) said media comprises penetrative means capable of providing an array of microchannels extending through partially or completely an epidermis layer of the skin; and a photocurable or a heat-curable polymer in admixture with said penetrative means; optionally wherein said penetrative means comprises needle-like structures and/or said penetrative means is isolated from a natural source; ii) said media is formulated as a solid, liquid, or a mixture, preferably selected from a gel, paste, or an emulsion; iii) said media further comprises at least one compound intended to be transported across the epidermis layer of said skin; or iv) said media is coupled to a stimulation means, said stimulation means being configured to perform at least one transdermal delivery technique selected the group consisting of heating, application of electric field, physical agitation, iontophoresis, electroporation, and electrophoresis; optionally wherein said stimulation means is configured to perform a combination of iontophoresis and electroporation;
and
(b) administering, to said skin, transdermal delivery technique comprising a combination of iontophoresis and electroporation,
wherein steps (a) and (b) are performed sequentially, or concurrently.

15. The method of claim 14, wherein said step (b) comprises administering to the skin at least one further transdermal delivery technique selected from the group consisting of heating, ultra-sound, laser dermabrasion, air jetting, water jetting, electrophoresis, and electro-osmosis.

16. A device for transporting a compound across a skin layer, said device comprising:
(i) a media for application onto the surface of said skin layer, said media comprising the compound to be transported, and a plurality of penetrative means configured to puncture said skin layer to thereby provide an array of microchannels extending partially or completely through an epidermis layer of the skin; and
(ii) a stimulation means coupled to said skin or said media, said stimulation means configured to apply to said skin or media at least one transdermal delivery technique selected from the group consisting of heating, ultra-sound, laser dermabrasion, air jetting, water jetting, electrophoresis, electro-osmosis, iontophoresis, and electroporation.

17. The device of claim 16, wherein the stimulation means is configured to apply a combination of iontophoresis and electroporation.

18. The device of claim 16, wherein the media further comprises a photocurable or a heat-curable polymer in admixture with said penetrative means.

19. The device of claim 16, further comprising at least one control means communicated with said stimulation means, for adjusting the strength, intensity, and/or duration of said transdermal delivery technique.

20. The device of claim 16, further comprising at least one compound source being fluidly communicated with said media or said skin, said compound source being configured to deliver said compound to the media, or to the surface of the skin layer, continuously or batch-wise.

21. The device of claim 16, wherein a) said media comprises penetrative means capable of providing an array of microchannels extending through partially or completely an epidermis layer of the skin; and a photocurable or a heat-curable polymer in admixture with said penetrative means; optionally wherein said penetrative means comprises needle-like structures and/or said penetrative means is isolated from a natural source; b) said media is formulated as a solid, liquid, or a mixture, preferably selected from a gel, paste, or an emulsion; c) said media further comprises at least one compound intended to be transported across the epidermis layer of said skin; or d) said media is coupled to a stimulation means, said stimulation means being configured to perform at least one transdermal delivery technique selected the group consisting of heating, application of electric field, physical agitation, iontophoresis, electroporation, and electrophoresis; optionally wherein said stimulation means is configured to perform a combination of iontophoresis and electroporation.
